(19) United States
(12) Patent Application Publication
(10) Pub. No.: US 2021/0340475 A1
(43) Pub. Date: Nov. 4, 2021

(54) Apparatus for cell culture and method for cell culture using the same

(71) Applicants: POSTECH Research and Business Development Foundation, Pohang-si (KR); Board of Regents, The University of Texas System, Austin, TX (US)

(72) Inventors: Dong Sung KIM, Pohang-si (KR); Woojung Shin, Austin, TX (US); Hyun Jung Kim, Austin, TX (US); Jae Seung Yoon, Pohang-si (KR); Hyeon Beom Chong, Pohang-si (KR)

(21) Appl. No.: 17/238,332

(22) Filed: Apr. 23, 2021

Related U.S. Application Data
(60) Provisional application No. 63/017,134, filed on Apr. 29, 2020.

(51) Int. Cl. C12M 1/32; C12M 1/04; C12M 1/12; C12M 1/00; C12M 1/34

(52) U.S. Cl. CPC C12M 23/12; C12M 23/24; C12M 41/32; C12M 29/04; C12M 25/04

(57) ABSTRACT

The present disclosure provides a three-dimensional cell culture method and a cell culture apparatus using the same. The cell culture method includes lowering, when culturing cells on at least one surface of a porous support, a concentration of a cell-secreted substance in a lower region of the other surface of the porous support. The cell culture apparatus includes: an upper chamber including an opening, porous support having at least one surface on which cells are cultured, and a cell culture space in which a culture medium is housed; a lower chamber including a space in which at least one upper chamber is disposed; and a device lowering a concentration of a cell-secreted substance in a lower region of the other surface of the porous support.

### CROSS-REFERENCE TO RELATED APPLICATION(S)

This application claims benefit of priority to U.S. Provisional Patent Application No. 63/017,134 filed on Apr. 29, 2020 in the Korean Intellectual Property Office, the disclosure of which is incorporated herein by reference in its entirety.

### BACKGROUND

### 1. Field

The present disclosure relates to a three-dimensional cell culture method and a cell culture apparatus using the same, and more particularly, to a cell culture apparatus capable of culturing cells in a form similar to tissues in vivo, for example, similar to a structure of intestinal epithelial cells in vivo, and a cell culture method using the same.

### 2. Description of Related Art

A concept of a microfluidic-based lab-on-a-chip technology has been developed to an organ-on-a-chip, and has the potential to be applied to a new platform for a drug response test. Accordingly, recently, many researchers are actively conducting research on an optimized design suitable for specific purposes by utilizing the microfluidic technology. This technology may provide quantitative information on drug screening and drug delivery systems by implementing the properties and functions of different organs in vitro and simulating an interaction between cells and the surrounding microenvironment. In order to form tissues similar to tissues in vivo in a laboratory, it is required to induce animal cells to have a three-dimensional structure when culturing the animal cells.

Meanwhile, an intestinal tissue model among various organs in vivo is a representative in vitro animal tissue model widely used in drug evaluation and new drug development. The intestinal tissue model plays an important role in allowing absorption, metabolism, and the like of drugs in the intestine to be simulated, observed, and analyzed in vitro. In general, an intestinal tissue model formed based on a commercially available cell culture insert has been utilized for drug evaluation.

However, actual intestinal tissues in vivo have a three-dimensional villi structure, whereas intestinal cells cultured in the insert as described above have no three-dimensional villi structure and are only cultured while maintaining a two-dimensional structure. Therefore, a biochemical phenomenon and a reaction caused by external stimuli occurring in a two-dimensional intestinal tissue model according to the related art are different from those in vivo. This results in significantly lowering the reliability and effectiveness of research on microbial behavior and influence, drug evaluation, and drug candidate screening in the in vitro intestinal tissue model. Therefore, it is very difficult to culture intestinal cells to have a three-dimensional structure and physiological functions similar to those of intestinal tissues in vivo.

### SUMMARY

An aspect of the present disclosure may provide a cell culture apparatus and method capable of inducing a three-dimensional structure when culturing cells, and more particularly, may provide a cell culture method capable of inducing animal cells to have a three-dimensional structure, and in particular, to have a three-dimensional villi structure when culturing the animal cells in vitro by implementing a physical environment such as regulation of cellular secretory substances that affect the formation of a three-dimensional structure and formation of a flow for the regulation when culturing cells, and a cell culture apparatus using the same.

According to an aspect of the present disclosure, a cell culture method may include lowering, when culturing cells on at least one surface of a porous support, the concentration of a cell-secreted substance in a lower region of the other surface of the porous support.

According to another aspect of the present disclosure, a cell culture apparatus may include an upper chamber including an opening, porous support having at least one surface on which cells are cultured, and a cell culture space in which a culture medium is housed; a lower chamber including a space in which at least one upper chamber is disposed; and a device lowering a concentration of a cell-secreted substance in a lower region of the other surface of the porous support.

### BRIEF DESCRIPTION OF DRAWINGS

The patent or application file contains at least one drawing executed in color. Copies of this patent or patent application publication with color drawing(s) will be provided by the Patent and Trademark Office upon request and payment of the necessary fee. The above and other aspects, features, and advantages of the present disclosure will be more clearly understood from the following detailed description taken in conjunction with the accompanying drawings, in which:

FIG. 1A illustrates an existing cell culture apparatus, and FIG. 1B illustrates a cell culture apparatus according to an exemplary embodiment in the present disclosure;

FIG. 2A illustrates a result of an influence of various types of antagonists acting on a Wnt signaling pathway on intestinal tissue morphogenesis, and FIGS. 2B and 2C illustrate experimental results of an influence of DKK-1 on intestinal cell morphogenesis;

FIGS. 3A through 3C illustrate an influence of intestinal tissue morphogenesis according to the presence of a flow of a culture medium applied to an apical side (AP) and/or a basolateral side (BL) based on a cell culture layer when culturing intestinal cells, in which FIG. 3A illustrates a result of observing the morphology of cells formed in a case where the flow of the culture medium is applied to both AP and BL, FIG. 3B illustrates a result of observing the morphology of cells formed in a case where the flow of the culture medium is applied to only BL, and FIG. 3C illustrates a result of observing the morphology of cells formed in a case where the flow of the culture medium is applied to only AP;

FIGS. 4A through 4C illustrate results of comparing the effects of reducing a concentration of a cell-secreted substance in a cell culture apparatus according to the related art and a cell culture apparatus according to the present disclosure, in which FIG. 4A illustrates a case where a motion of a fluid is not applied, FIG. 4B illustrates a case where a motion of a fluid is applied, and FIG. 4C illustrates a case where an attempt is made to apply a motion of a fluid, but the motion of the fluid is not substantially generated because a mass of the fluid contained in a lower chamber is insufficient;

FIGS. 5A through 5C illustrate an influence of a rotational motion of fluid on intestinal tissue morphogenesis, in which FIG. 5A illustrates a case where a rotational motion of a fluid is not applied, FIG. 5B illustrates a case where an attempt is made to apply a rotational motion of a fluid, but the rotational motion of the fluid is not substantially generated in a lower chamber, and FIG. 5C illustrates a case where a rotational motion of a fluid is applied;

FIGS. 6A through 6C illustrate results of comparing a function of intestinal tissues having a three-dimensional villi structure by a rotational motion of a fluid cultured in the cell culture apparatus according to the present disclosure with a function of intestinal tissues cultured in the cell culture apparatus according to the related art, in which FIG. 6A illustrates a result of observing a percentage of proliferative intestinal cell nuclei (Ki67), FIG. 6B illustrates a result of observing the expression of a metabolic protein (CYP3A4), and FIG. 6C illustrates a result of observing the expression of a protein (MUC2) related to mucus secretion of intestinal cells; and

FIGS. 7A through 7C illustrate cell culture apparatuses according to exemplary embodiments in the present disclosure, in which FIG. 7A illustrates a Gut-on-a-chip, FIG. 7B illustrates a cell culture apparatus configured to allow fluid to flow, and FIG. 7C illustrates a cell culture apparatus configured to apply a motion of a fluid.

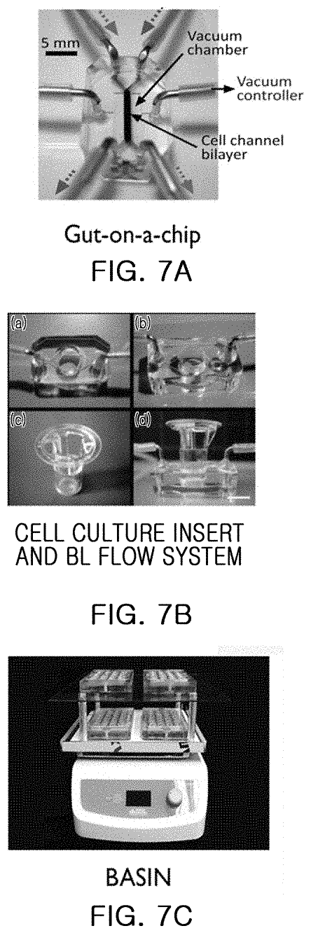

### DETAILED DESCRIPTION

Hereinafter, exemplary embodiments in the present disclosure will now be described in detail with reference to the accompanying drawings. However, exemplary embodiments in the present disclosure may be modified in several other forms, and the scope of the present disclosure is not limited to exemplary embodiments to be described below.

The present disclosure investigates factors that play an important role in the morphogenesis of animal tissues and the effects thereof. The present disclosure may provide a cell culture apparatus and method capable of inducing morphogenesis of animal tissues by controlling corresponding factors. In particular, the present disclosure may provide an apparatus and method capable of inducing the formation of a three-dimensional villi structure when culturing epithelial cells such as intestinal epithelial cells and implementing mass cultivation of epithelial cells.

Specifically, a cell culture method according to the present disclosure may include lowering, when culturing cells on at least one surface of a porous support, a concentration of a cell-secreted substance in a lower region of the other surface of the porous support.

The lower region of the other surface of the porous support may include a region spaced apart from the porous support by 0.01 mm, and may refer to, for example, a region between the porous support and a position spaced apart from the porous support by 0.1 mm, 0.5 mm, 1 mm, 2 mm, 3 mm, or the like while including the region spaced apart from the porous support by 0.01 mm on a surface opposite to a surface on which cells are cultured. However, the present disclosure is not limited thereto. The lower region of the other surface of the porous support may be a region that includes the above region and is larger than the above region. It is not intended to exclude a region spaced apart from the porous support within 0.01 mm. A region spaced apart from the porous support within 0.01 mm, for example, 0.0001 mm or 0.005 mm, is also to be interpreted to be included in the “lower region of the other surface of the porous support” according to the present disclosure. Any region adjacent to the porous support may be included in the lower region of the other surface of the porous support. The lowering of the concentration of the cell-secreted substance in the region adjacent to the porous support as described above may be a precondition of the cell culture method according to the present disclosure.

The concentration of the cell-secreted substance in the lowering of the concentration of the cell-secreted substance may be maintained at 10 μg/ml or less, but is not limited thereto.

In the present disclosure, the lowering of the concentration of the cell-secreted substance in the lower region of the other surface of the porous support may be performed for a period of 10% or more, for example, 50% or more, preferably 80% or more, and more preferably 90% or more, of a total cell culture period, and the period may be continuous or discontinuous.

The cell-secreted substance may include at least one component selected from the group consisting of sFRP1, FRZB, Sizzled, Sizzled2, Crescent, WIF-1, Cerberus, Coco, DKK-2, DKK-3, DKK-4, Soggy, sFRP2, sFRP3, sFRP4, sFRP5, and DKK-1, and may preferably include DKK-2. More preferably, in the lowering of the concentration of the cell-secreted substance, a concentration of DKK-1 may be maintained at 10 μg/ml or less.

In the present disclosure, the lowering of the concentration of the cell-secreted substance in the lower region may be performed by i) supplying a flowing fluid to the lower region, ii) applying a motion of fluid to the lower region, or iii) adding an antibody against the cell-secreted substance.

More preferably, in i) the supplying of the flowing fluid to the lower region, a fluid may be allowed to inflow and outflow into a basolateral side (BL) of a cell culture layer including the lower region to form a flow of the fluid in at least one direction.

Meanwhile, in ii) the applying of the motion of the fluid to the lower region, a fluid does not flow as described above, but a movement of the fluid in a static state is also included. For example, the motion of the fluid may be applied by at least one selected from the group consisting of a reciprocating motion of a fluid, a rotational motion of a fluid, vortex formation of a fluid, a gradient motion of a fluid, and a random motion of a fluid, and may include a vertical motion and/or a horizontal motion of a fluid. However, the present disclosure is not limited thereto. The motion of the fluid may include all motions of a fluid for rapidly diffusing, diluting, and separating the cell secreted substance secreted by cells in culture in the lower region.

Meanwhile, it is possible to lower the concentration of the cell-secreted substance secreted by cells in the lower region of the other surface of the porous support by increasing the volume of the fluid in the lower region to induce simple diffusion of the cell-secreted substance into BL of the cell culture layer without applying the flow and motion of the fluid. However, in this case, the flow and motion of the fluid are not smooth as compared to a case where the three-dimensional structure such as villi-like protrusions is formed to apply the flow and motion of the fluid, and the density of protrusions may be low or a distribution of positions where the protrusions are formed is not uniform.

In addition, the present disclosure may provide a cell culture apparatus capable of implementing the cell culture method according to the present disclosure.

More specifically, the cell culture apparatus according to the present disclosure may include an upper chamber including an opening, porous support having at least one surface on which cells are cultured, and a cell culture space in which a culture medium is housed; a lower chamber including a space in which at least one upper chamber is disposed; and a device lowering a concentration of a cell-secreted substance in a lower region of the other surface of the porous support.

In this case, the lower region of the other surface of the porous support may include a region spaced apart from the porous support by 0.01 mm, and may be the same as described in the cell culture method.

In the cell culture apparatus according to the present disclosure, the upper chamber may be, for example, a Transwell insert for cell culture used in the art. In this case, the upper chamber may be referred to interchangeably with the Transwell insert, and the porous support coupled to the upper chamber may be a thin membrane having a pore structure and a thickness of 1 mm or less.

The porous support may include a plurality of pores, and may be, for example, a porous support having a porosity of 2% to 60% or a porous support having a porosity of 5% to 20%. When the porosity is less than 2%, a water permeability coefficient may be lowered, and substance diffusion through the porous support may thus be deteriorated, which causes a reduction in the survival rate of the cells in culture on the porous support.

The porous support may be formed by a nanofiber network, may be formed by randomly entangling a plurality of nanofibers, or may be formed by molding a synthetic polymer resin. The porous support is formed by a network of a plurality of polymer nanofibers, such that the porous support may have a structure similar to that of a basal membrane in vivo. Therefore, it is possible to provide an environment similar to a biological microstructure environment.

The porous support may include a nanofiber network having nanoscale pores, microscale pores, and a combination thereof, a porous thin membrane, or a combination thereof, and may include a plurality of pores. In this case, the pore may have a size of several nanometers to several hundred micrometers. That is, the porous support may serve as a selectively permeable membrane through which substances such as nutrients and growth factors in a cell culture medium selectively permeate without permeation of single cells because the porous support has the pores. Therefore, the porous support may serve as a substance transfer barrier or passage. For example, the porous support may have a pore size of an average diameter of 10 nm to 100 μm, and preferably 10 nm to 8 μm.

In this case, the polymer nanofiber or the synthetic polymer resin may include at least one of thermoplastic resin, a thermosetting resin, an elastomer, and a biopolymer. For example, the polymer nanofiber or the synthetic polymer resin may include at least one of silk fibroin, polycaprolactone, polyurethane, polyvinylidene fluoride (PVDF), polystyrene, collagen, gelatin, and chitosan, but the present disclosure is not limited thereto.

The porous support of the upper chamber may be produced by using a mold in which a depressed pattern is formed to be concave downward. For example, the porous support may be produced by placing a flat porous membrane formed of a polymer nanofiber on the mold and pressurizing the porous membrane with another mold for an embossing process, but the present disclosure is not limited thereto. In this case, an upper surface of the porous support of the upper chamber, which is a region serving as a cell culture layer, is formed to be concave to form a cell culture space in which a culture medium is housed. Therefore, cells may be easily seated in the cell culture layer, may be cultured in a three-dimensional structure, and may be stably proliferated or differentiated.

The cell culture apparatus according to the present disclosure may include the lower chamber. The lower chamber may include the space in which at least one upper chamber is disposed and may be configured to implement a flow and/or a motion of a fluid by the device lowering the concentration of the cell-secreted substance in the lower region of the other surface of the porous support.

For example, in a chamber according to the related art in which a plurality of Transwell inserts are disposed, the respective Transwell inserts are disposed so that they are separately partitioned. However, in this case, the cell-secreted substance cannot be separated or diffused by the motion of the fluid. Therefore, the object of the present disclosure cannot be achieved. Accordingly, in the cell culture apparatus according to the present disclosure, a volume of the lower chamber after the upper chamber is disposed in the lower chamber may be preferably 1.5 times or more, for example, 2 times to 1,000 times the volume of the upper chamber.

That is, in the cell culture apparatus according to the present disclosure, a distance between the porous support of the lower chamber and the bottom of the lower chamber after the upper chamber is disposed in the lower chamber may be preferably 1 mm, 2 mm, 3 mm, or the like, but the present disclosure is not particularly limited thereto. However, the fluid in the lower region of the porous support is moved by the flow and/or the motion of the fluid, even in a case where the distance is smaller than the above distance. Therefore, the lower chamber may have a side volume at which the cell-secreted substance present in the region adjacent to the porous support may be rapidly diffused, diluted, and separated.

As described above, the device lowering the concentration of the cell-secreted substance in the lower region of the other surface of the porous support may be i) a device supplying a flowing fluid to the lower chamber or ii) a device applying a motion of fluid to the lower chamber.

More specifically, the device applying the motion of the fluid may induce the flow of the fluid by moving the lower chamber in a specific or random trajectory and/or direction. For example, the device applying the motion of the fluid may move the lower chamber in a horizontal line, but the motion is not limited to the above motion. For example, the device applying the motion of the fluid may include at least one device selected from the group consisting of a shaker such as an orbital shaker, a waving shaker, or a locking shaker, an agitator, and an ultrasonic device, but the present disclosure is not limited thereto. A device capable of implementing a movement of a fluid in a static state may be used without limitation.

For example, the motion of the fluid generated by the device may be at least one selected from the group consisting of a reciprocating motion of a fluid, a rotational motion of a fluid, vortex formation of a fluid, a gradient motion of a fluid, and a random motion of a fluid, and may include a vertical motion and/or a horizontal motion of a fluid, but the present disclosure is not limited thereto. The motion of the fluid may include all motions of fluid for rapidly diffusing, diluting, and separating the cell-secreted substance secreted by cells in culture in the lower region.

The device supplying the flowing fluid to the lower chamber may include a fluid inlet and a fluid outlet provided in the lower chamber; and a device inducing a flow of the fluid from the fluid inlet to the fluid outlet, but the present disclosure is not limited thereto. Any device may be used as long as it has a structure capable of allowing the fluid to flow.

In addition, the device inducing the flow of the fluid may be additionally connected to the lower chamber. For example, the lower chamber may be connected to the device inducing the flow of the fluid to allow the fluid to flow in a horizontal direction. In this case, a pump may be used as the device inducing the flow of the fluid, but any device may be used as long as it may induce a flow of the fluid. For example, a syringe pump or a tube peristaltic pump may be used as the device, and any device may be used as long as it may induce or promote a movement of the fluid.

In this case, the fluid may flow in one direction or several directions, but the direction is not particularly limited as long as the flow of the fluid exists.

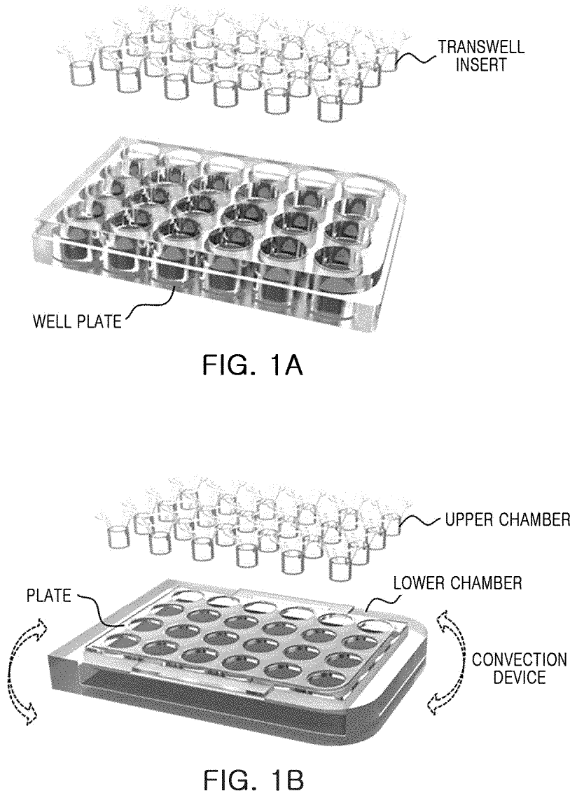

In addition, the cell culture apparatus according to the present disclosure may further include a plate through which at least one upper chamber penetrates to be inserted and fixed thereto, and the plate may have a shape in which a plurality of upper chambers may penetrate to be inserted thereto. More specifically, as illustrated in FIG. 1B, the plurality of the upper chamber is inserted into the plate, such that the upper chambers may be fixed at a position spaced apart from a bottom surface of the lower chamber by a certain distance.

In this case, one or more upper chambers may be inserted into the plate, and a penetration portion may be formed to correspond to the number of upper chambers. For example, as in a well plate used in the art, a penetration portion into which 6, 12, 24, 48, 96, 128, or 256 upper chambers may be inserted and which corresponds to the plate may be formed.

The fluid which may be used in the cell culture method and the cell culture apparatus according to the present disclosure may be a culture medium, a phosphate-buffered saline (PBS), or a mixture thereof, and may be preferably a cell culture medium. FBS, glucose, and growth factors may be included in the cell culture medium, and any cell culture medium may be used as long as it is a cell culture medium used in the art.

Cells which may be applied to the cell culture method and the cell culture apparatus according to the present disclosure are not particularly limited, and may be epithelial cells of an animal including a human. For example, the cells may be epithelial cells of gastrointestinal system such as intestinal epithelial cells. More specifically, the intestinal epithelial cells may be human cells, and may be primary cells, primary small intestinal cells, primary large intestinal cells, small intestinal cells, large intestinal cells, cultured cells, passaged cells (subcultured cells), immortalized cells, transgenic cells, genetically modified cells, cancerous cells or cells from an animal with intestinal cancer, cells from an animal with an intestinal disease or disorder, stem cells, embryonic stem cells (ESCs), induced pluripotent stem cells (iPSCs), Paneth cells, crypt cells, mucus-secreting cells, Caco-2 cells, HT-29 cells, intestinal organoids, or cells derived from intestinal organoids.

The intestinal epithelial cells may form a three-dimensional protrusion structure, that is, a villi-like structure, in vivo. Although culturing in vitro is performed in the cell culture apparatus according to the present disclosure, the intestinal epithelial cells may form a structure similar to that of the intestinal epithelial cells in vivo.

In intestinal tissue morphogenesis, the intestinal tissue morphogenesis is inhibited by a cell-secreted substance including a substance that inhibits morphogenesis-related signaling. The cell-secreted substance may be produced in a lower region of intestinal tissue cells and may act on the intestinal tissue cells. As in the present disclosure, in a case where the flow and/or the movement of the fluid is smooth by the formation of the flow of the fluid in the lower region of the porous support in which the cells are cultured and the like, or in a case where a content of the cell-secreted substance itself is controlled by antibodies and the like, the factors that inhibit the growth of the cells are removed. As a result, the action of the cell-secreted substance on the intestinal tissue cells is removed. Therefore, it is possible to culture cells having a structure similar to that of cells in vivo.

The antibodies against the cell-secreted substance are not particularly limited, and may be produced using a technology well known in the art.

In the present disclosure, the cell-secreted substance may include a substance that inhibits morphogenesis-related signaling, and may include, for example, substances that inhibit Wnt signaling, such as sFRP1, FRZB, Sizzled, Sizzled2, Crescent, WIF-1, Cerberus, Coco, DKK-2, DKK-3, DKK-4, Soggy, sFRP2, sFRP3, sFRP4, sFRP5, and DKK-1.

The present disclosure may provide a cell culture method including, by using the cell culture apparatus according to the present disclosure, seeding cells onto the porous support of the upper chamber; and supplying a culture medium. The same contents described in the cell culture apparatus may also be applied to the cell culture method.

In this case, the cells may be cultured by being seeded onto the porous support through the seeding of the cells onto the porous support of the upper chamber. The seeding may be performed by seeding cells to be cultured onto the porous support prior to supplying a fluid, that is, a culture medium to the upper chamber.

According to the cell culture method and the cell culture apparatus according to the present disclosure, a three-dimensional structure may be formed by culturing cells for about 5 to 9 days. In the present disclosure, the lowering of the concentration of the cell-secreted substance in the lower region of the other surface of the porous support may be performed for a period of 10% or more, for example, 50% or more, preferably 80% or more, and more preferably 90% or more, of a total cell culture period, and the period may be continuous or discontinuous.

Hereinafter, the present disclosure will be described in more detail with reference to specific examples. The following examples are only examples provided in order to assist in the understanding of the present disclosure, but the scope of the present disclosure is not limited thereto.

### Examples

1. Detection of Factors Effective in Intestinal Tissue Morphogenesis

(1) Influence of Various Types of Antagonists Acting on Wnt Signaling Pathway on Intestinal Tissue Morphogenesis

1×10⁷ cells/mL of Caco-2 cells were seeded onto an upper microchannel of a Gut-on-a-chip culture apparatus formed of a silicone polymer, and each of untreated (control), recombinant DKK-1 (rDKK-1), recombinant Wnt inhibitory factor 1 (rWIF-1), recombinant secreted frizzled-related protein 1, (rsFRP-1), and recombinant Soggy-1/DKK-like 1 (rSoggy-1/DKKL-1) was treated at a concentration of 100 ng/mL while applying a flow (30 μL/h) to a lower microchannel, the cells were cultured at 37° C. for 48 hours, and the culture results were observed.

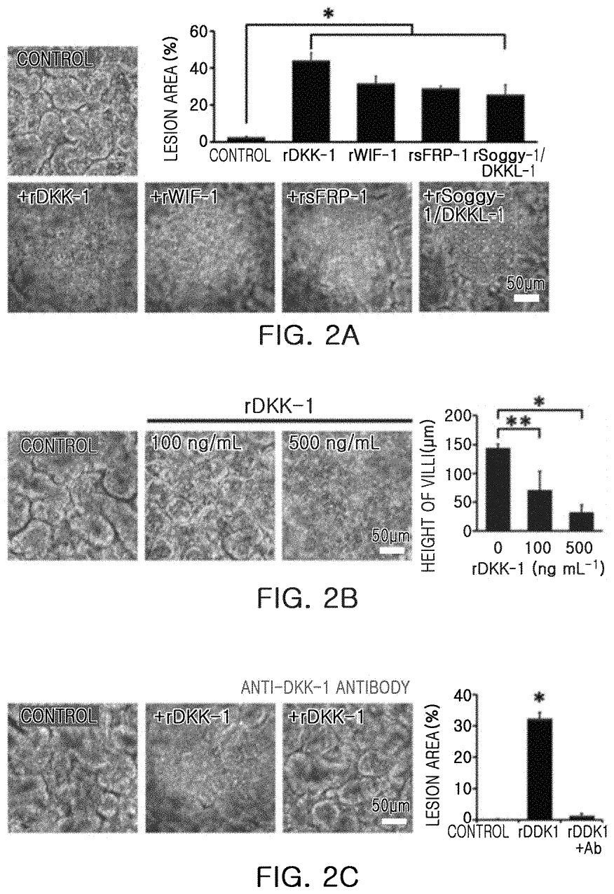

As a result, as illustrated in FIG. 2A, it could be confirmed that in the case where rDKK-1 was treated, a lesion area was the largest.

(2) Inhibition of Intestinal Tissue Morphogenesis According to Concentration of rDKK-1

1×10⁷ cells/mL of Caco-2 cells were seeded and attached onto each of upper microchannels of three different culture apparatuses to form a two-dimensional single mucosal surface, each of untreated (control) and 100 ng/mL and 500 ng/mL of recombinant DKK-1 (rDKK-1) was treated while applying a flow (30 μL/h) to each of lower microchannels, the cells were cultured at 37° C. for 48 hours, and the culture results were observed.

As a result, as illustrated in FIG. 2B, it could be confirmed that three-dimensional intestinal tissue morphogenesis was suppressed as the concentration of rDKK-1 was increased, resulting in a low height of the formed villi. An exemplified apparatus according to the present disclosure illustrated in FIG. 2B is referred to as a “Basolateral convective flow-generating multi-well inserts platform (BASIN)”.

(3) Whether or not Intestinal Tissue Morphogenesis was Suppressed in Inhibition by DKK-1

1×10⁷ cells/mL of Caco-2 cells were seeded onto each of upper microchannels of three different culture apparatuses, each of untreated (control), 500 ng/mL of recombinant DKK-1 (rDKK-1), and 500 ng/mL of rDKK-1 and 20 ng/mL of anti-rDKK-1 antibodies was treated, the cells were cultured at 37° C. for 48 hours, and the culture results were observed.

As a result, as illustrated in FIG. 2C, it could be confirmed that in a case where rDKK-1 was treated together with rDKK-1 antibodies, the intestinal tissue morphogenesis was suppressed by DKK-1. Therefore, it could be confirmed that the three-dimensional intestinal cell morphogenesis was suppressed by DKK-1.

2. Cell Culture Experiment with Flow of Fluid

1×10⁷ cells/mL of Caco-2 cells were seeded into a fluidic Gut-on-a-chip culture apparatus in which an apical side (AP) and a basolateral side (BL) were divided by a porous silicone membrane, and a flow of the culture medium was applied to each of AP and BL based on a cell culture layer in which Caco-2 cells were cultured. In addition, in order to simulate intestinal peristalsis-like mechanical deformation, a vacuum controller (FlexCell) was connected to a vacuum chamber formed inside the Gut-on-a-chip, a flow condition (10% cell stretching, 0.15 Hz frequency) optimized to simulate the intestinal peristalsis was applied, and then the same repetitive flow motion condition as the Sine function was applied.

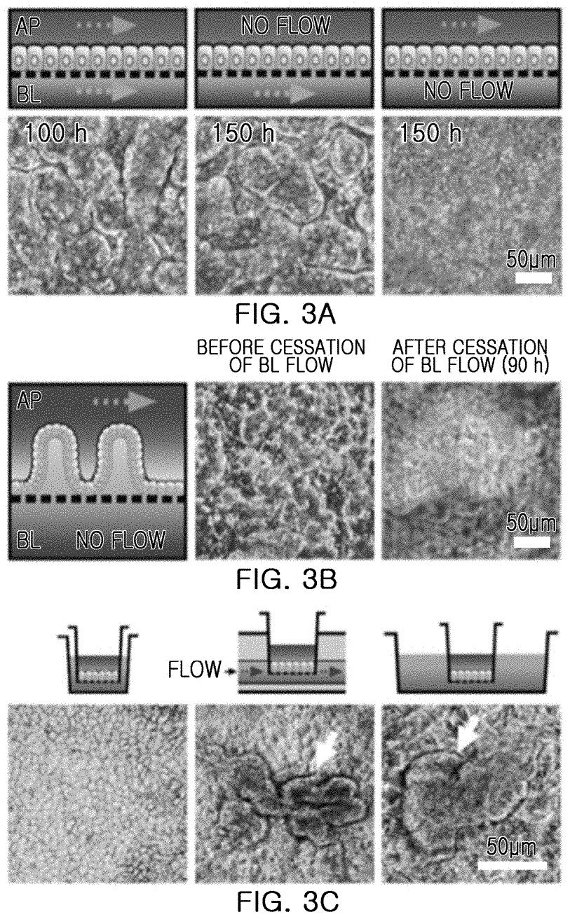

The intestinal tissue morphogenesis formed in each of the case where the flow (30 μL/h) of the culture medium was applied to both AP and BL at 37° C. (culture was performed for 100 hours), the case where the culture medium was applied to only BL (culture was performed for 150 hours), and the case where the culture medium was applied to only AP (culture was performed for 150 hours) was observed. The results are illustrated in FIG. 3A through 3C. In FIG. 3A through 3C, the region indicated by the arrow means that the flow is applied.

As a result, as illustrated in FIG. 3A, it could be confirmed that the intestinal tissue morphogenesis similar to the intestinal tissue morphogenesis in vivo was formed, through both the left and middle photographs of FIG. 3A obtained by applying the flow of the culture medium to both AP and BL.

Further, in the case where the flow of the culture medium was applied to AP, after the morphogenesis similar to intestinal tissue morphogenesis in vivo was formed, the flow of the culture medium was not applied to BL for 90 hours, and the culture result was observed again. The results are illustrated in FIG. 3B.

As a result, as illustrated in FIG. 3B, it could be confirmed that in the case where the flow of the culture medium to BL was ceased, the three-dimensional structure was disrupted over time even in the cells in which the three-dimensional morphogenesis similar to the intestinal tissues in vivo was formed.

In addition, 5×10⁵ cells/mL of Caco-2 cells were seeded into a cell culture nanoporous insert used in the art in which upper and lower chambers were divided with a porous membrane, and each of the flow and the diffusion was applied to only BL based on the cell culture layer in which the Caco-2 cells were cultured to culture intestinal cells. As the type used in the art, the upper chamber into which the cells were seeded was divided into the case where a limited capacity of fluid was in a static state on BL (the left photograph of FIG. 3C), the case where a flow was applied to only BL (the middle photograph of FIG. 3C), and the case where diffusion was applied to only BL (the right photograph of FIG. 3C), and the cells were cultured. The results are illustrated at the bottom of each of the cases of FIG. 3C. In this case, as the type used in the art, in the case as illustrated in the left photograph of FIG. 3C, 500 μm of fluid was used in the lower chamber, and in the case where the diffusion was applied to BL as illustrated in the right photograph of FIG. 3C, the upper chamber was installed in a 70 mL large-capacity chamber, and the cells were cultured for up to 120 hours.

As a result, as illustrated in FIG. 3C, it could be confirmed that even in the case where the flow and the diffusion were applied, the morphogenesis similar to the intestinal tissue morphogenesis in vivo was induced. However, in the case where the flow was applied to BL as illustrated in the middle photograph of FIG. 3C, villi-like projections were formed by applying the flow for 48 hours, whereas in the case where the diffusion was applied to only BL as illustrated in the right photograph of FIG. 3C, the density of the projections was low or the distribution of the positions where the protrusions were formed was not uniform.

In the case of the experiment from which the results of FIG. 3A were derived, conditions for villi formation of the intestinal epithelial cells were realized by precisely controlling the flow rate and the flow of the fluid through a computer manufacturing technology and a microchip manufacturing technology. On the other hand, in the case of the experiment from which the results of FIG. 3C were derived, the intestinal villi formation was induced by removing the cell-secreted substance including DKK-1 and minimizing the concentration thereof, even though the nanoporous insert commonly used in the art was used. However, in the latter case, the density of the projections was low or the distribution of the positions where the protrusions were formed was not uniform.

The culture process based on the nanoporous insert-based static culture well plate commonly used in the art may be improved through such findings in the future. Therefore, it is possible to derive the advantages of freely inducing the three-dimensional intestinal villi formation and implementing various complex system functions without the introduction of complex mechanical apparatuses.

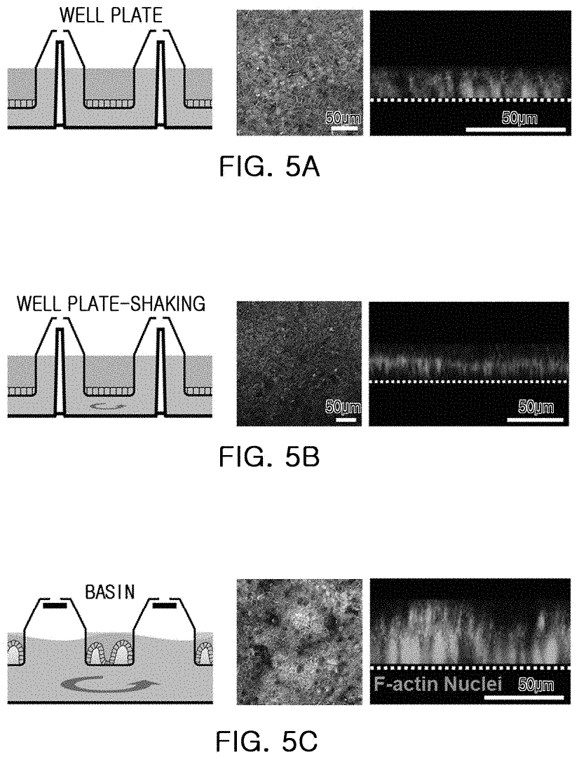

As a result, it could be confirmed that in the case where Caco-2 cells were cultured in the apparatus of FIG. 1A which was a general cell culture apparatus, the morphogenesis with the 2D structure was formed as illustrated in FIG. 5A, whereas in the case where Coco-2 cells were cultured by applying the flow of the fluid in the apparatus of FIG. 1B, the morphogenesis with the three-dimensional structure similar to the intestinal tissue cells in vivo was induced as illustrated in FIG. 5C.

3. Observation of Characteristics of Cells Cultured According to the Present Disclosure

Porous inserts commonly used in the art were fixed to a lower chamber with no partitions of the BASIN culture apparatus according to the present disclosure, 5×10⁵ cells/mL of Caco-2 cells were seeded, and a flow of a culture medium was applied to BL based on a cell culture layer in which the Caco-2 cells were cultured using an orbital shaker (30 rpm) referred to as MaXshaker. In addition, 5×10⁵ cells/mL of Caco-2 cells were seeded into a cell culture porous insert used in the art in which upper and lower chambers were divided by a porous membrane, and intestinal cells were cultured in a static state.

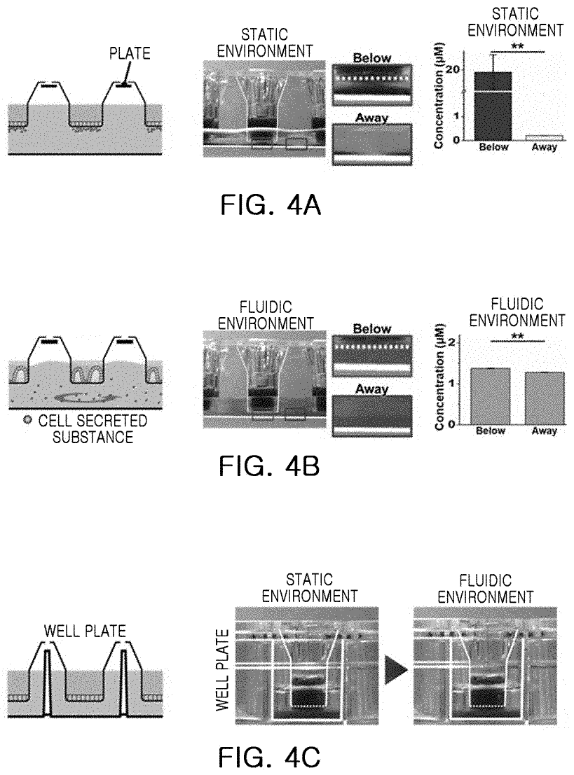

As a result, as illustrated in FIG. 4B, the flow of the fluid contained was more effectively induced by the lower chamber with no partitions of the cell culture apparatus according to the present disclosure, unlike a commercially available Transwell plate. Therefore, the movement of the substance in the fluid through convection and the accompanying mixing were induced. The features of such an apparatus may lead to the effect of lowering the concentration of the cell secreted substance in the lower region of the porous support of the upper chamber.

As illustrated in FIGS. 5A and 5B, it could be confirmed that the Caco-2 cells were cultured in a flat 2D single cell layer form without formation of the 3D villi structure not only in the general Transwell plate in which the motion of the fluid was not applied but in the Transwell plate operated on the orbital shaker, whereas the 3D villi structure was formed in the Caco-2 cells cultured in the BASIN apparatus according to the present disclosure as illustrated in FIG. 5C. The middle and right photographs of FIGS. 5A through 5C illustrate the results of observing cytoskeletal stain from the above (middle photograph) and side (right photograph) after the Caco-2 cells were cultured in the upper chamber (Transwell insert) for 5 days to 9 days.

In order to evaluate functional improvements of the Caco-2 cells cultured in the BASIN according to the present disclosure, the cells were cultured for 100 hours, fluorescence staining of each of a Ki67 marker, a CYP3A4 marker, and a MUC2 marker was performed, intestinal cells cultured in the static Transwell plate as illustrated in FIG. 5A were selected as a control group, and the comparison was carried out.

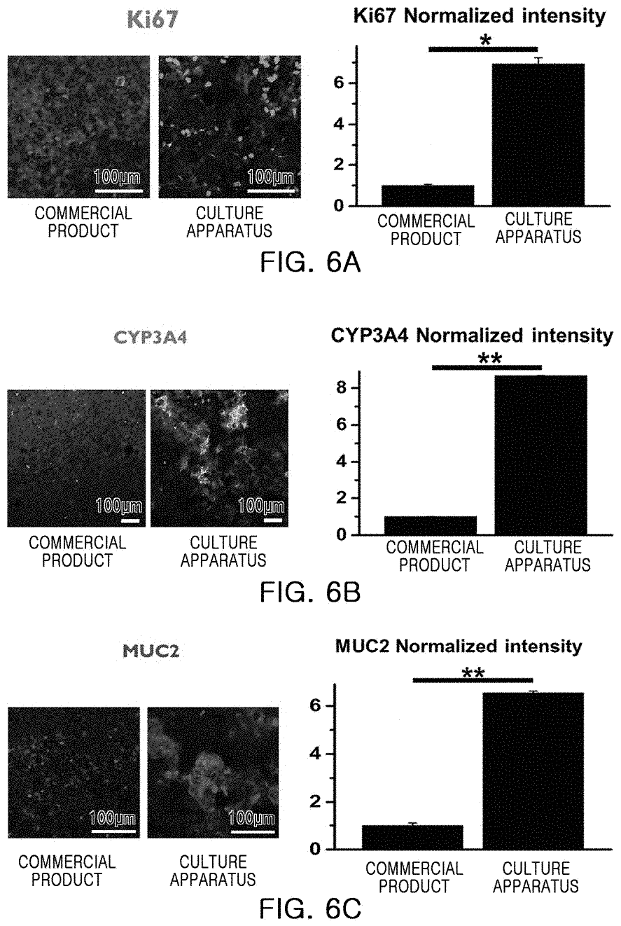

As a result, it could be confirmed that in the case of the cell culture method according to the present disclosure, as illustrated in FIGS. 6A through 6C, the percentage of the proliferative intestinal cell nuclei (Ki67) was increased, the metabolic protein (CYP3A4) was more expressed, and further, the protein (MUC2) related to mucus secretion of the intestinal cells were more expressed.

As set forth above, in the case where the cells were cultured by the cell culture method and the cell culture apparatus according to the present disclosure, the secreted substance that cells secrete themselves may be efficiently regulated when culturing cells. Therefore, it is possible to induce the animal cells cultured in vitro to form a three-dimensional structure, for example, a three-dimensional villi structure that is the same as or similar to that of the cells in vivo when culturing intestinal epithelial cells. Therefore, in the field in which the intestinal tissue model is used, such as drug evaluation and new drug development, phenomena such as absorption and metabolism of drugs similar to phenomena occurring in an actual body may be simulated, observed, and analyzed in vitro.

While exemplary embodiments have been shown and described above, it will be apparent to those skilled in the art that modifications and variations could be made without departing from the scope of the present disclosure as defined by the appended claims.

What is claimed is:

1. A cell culture method comprising lowering a concentration of a cell-secreted substance in a lower region of the other surface of the porous support, when culturing cells on at least one surface of a porous support.

2. The cell culture method of claim 1, wherein the lower region of the other surface of the porous support includes a region spaced apart from the porous support by 0.01 mm.

3. The cell culture method of claim 1, wherein in the lowering of the concentration of the cell-secreted substance, the concentration of the cell-secreted substance is maintained at 100 μg/ml or less.

4. The cell culture method of claim 1, wherein the cell-secreted substance includes at least one component selected from the group consisting of sFRP1, FRZB, Sizzled, Sizzled2, Crescent, WIF-1, Cerberus, Coco, DKK-2, DKK-3, DKK-4, Soggy, sFRP2, sFRP3, sFRP4, sFRP5, and DKK-1.

5. The cell culture method of claim 1, wherein in the lowering of the concentration of the cell-secreted substance, a concentration of DKK-1 is maintained at 10 μg/ml or less.

6. The cell culture method of claim 1, wherein the lowering of the concentration of the cell-secreted substance in the lower region is performed by i) supplying a flowing fluid to the lower region, ii) applying a motion of fluid to the lower region, or iii) adding an antibody against the cell-secreted substance.

7. The cell culture method of claim 6, wherein the motion of the fluid is applied by at least one selected from the group consisting of a reciprocating motion of a fluid, a rotational motion of a fluid, vortex formation of a fluid, a gradient motion of a fluid, and a random motion of a fluid.

8. The cell culture method of claim 1, wherein the cell is an epithelial cell.

9. The cell culture method of claim 1, wherein the cell is an intestinal epithelial cell.

10. A cell culture apparatus comprising:
an upper chamber including an opening, a porous support having at least one surface on which cells are cultured, and a cell culture space in which a culture medium is housed;
a lower chamber including a space in which at least one upper chamber is disposed; and
a device lowering a concentration of a cell-secreted substance in a lower region of the other surface of the porous support.

11. The cell culture apparatus of claim 10, wherein the lower region of the other surface of the porous support includes a region spaced apart from the porous support by 0.01 mm.

12. The cell culture apparatus of claim 10, wherein the device lowering the concentration of the cell-secreted substance in the lower region of the other surface of the porous support is i) a device supplying a flowing fluid to the lower chamber or ii) a device applying a motion of fluid to the lower chamber.

13. The cell culture apparatus of claim 12, wherein the device applying the motion of the fluid is at least one device selected from the group consisting of a pump, a shaker, an agitator, and an ultrasonic device.

14. The cell culture apparatus of claim 12, wherein the device supplying the flowing fluid to the lower chamber includes:
a fluid inlet and a fluid outlet provided in the lower chamber; and
a device inducing a flow of the fluid from the fluid inlet to the fluid outlet.

15. The cell culture apparatus of claim 10, further comprising a plate through which at least one upper chamber penetrates to be inserted and fixed thereto.

16. The cell culture apparatus of claim 10, wherein the porous support includes a nanofiber network having nanoscale pores, microscale pores, and a combination thereof, a porous thin membrane, or a combination thereof.
